(19) United States
(12) Patent Application Publication
(10) Pub. No.: US 2003/0050705 A1
(43) Pub. Date: Mar. 13, 2003

(54) Acetabular cup

(75) Inventors: Christophe Cueille, Missy (FR); Denis Pichon, Mordelles (FR)

Correspondence Address: LERNER, DAVID, LITTENBERG,, KRUMHOLZ & MENTLIK, 600 SOUTH AVENUE WEST, WESTFIELD, NJ, 07090, US

(73) Assignee: Benoist Girard SAS, Herouville-saint-clair Cedex (FR)

(21) Appl. No.: 10/237,820

(22) Filed: Sep. 9, 2002

(30) Foreign Application Priority Data
Sep. 11, 2001 (GB) 0122002.9

(51) Int. Cl. A61F 2/32

(52) U.S. Cl. 623/22.24

(57) ABSTRACT

An acetabular cup for use in a total hip prosthesis employing a prosthetic femoral ball head sized to correspond to the anatomic head size of a natural femoral head has an inner bearing surface portion constructed from a ceramics material, an outer bone-interface layer, and an outer coating of a bone growth stimulant substance applied to the outer bone-interface layer.

### BACKGROUND OF THE INVENTION

This invention relates to an acetabular cup for a total hip prosthesis, a method of manufacture thereof, and a total hip prosthesis embodying such a cup.

A total replacement hip joint is comprised of two general prosthetic components which are implanted into the patient's hip and leg. A femoral ball component replaces the natural femoral head surface, and an acetabular cup component replaces the natural acetabular socket surface.

Femoral prosthetic components come in many different forms in order to suit the patient and to minimize the amount of natural bone which has to be removed. Some of these components are approximately hemispherical in shape and are fitted directly onto the femoral head. Other components comprise a stem portion which is implanted into the medullary canal, and a neck carrying a ball head, or a head spigot adapted to carry a ball head.

Acetabular cup components are also made in a variety of forms in order to suit the patient's needs. These components are generally hemispherical in shape and are fitted into the acetabulum.

It is important in a total replacement hip joint for the femoral head and the acetabular cup to have good bearing surfaces in order to provide a natural smooth hip movement. It is known to construct hip replacement components from metals such as titanium or stainless steel alloys, plastics materials such as polyethylene, and ceramics materials such as alumina.

However, the materials used must also be able to withstand loading of the joint post surgery. During natural gait and other natural movements various multi-directional loads are applied to the joint. In order to withstand these forces an acetabular cup must be either very strong, or attached uniformly to the acetabulum so the loads are dispersed across its spherical structure. It is known for prostheses to fail if they are not attached correctly to the bone because the loads become concentrated in small areas.

The most desired form of total hip prosthesis is a ceramics on ceramics arrangement with both the femoral ball head and the acetabular cup being constructed from a ceramics material. Further, it has been found that the bearing surface with a ceramics on ceramics arrangement works best with the known larger type of prosthetic ball heads and sockets which are of similar dimensions to the natural hip joint. These larger scale prosthetic joins also provide more stability during natural gait, and reduces the possibility of dislocation.

However, such an arrangement has not been thought possible because of the known difficulty in attaching a ceramic acetabular cup to the acetabulum. Ceramics materials are relatively brittle, and are known to fail without a uniform interface with the bone to distribute the load across the bone. Attempts have been made to attach larger size ceramic acetabular cups to the bone with cement and other known means, but they did not achieve a satisfactory attachment, which led to eventual failure when the load became concentrated in small areas. So far only ceramics acetabular cups provided with an outer metallic bone-interfacing shell have been successful. However, the outer layer increases the wall thickness of the cup, which reduces the interior space available for the bearing surface of the joint. Therefore, these acetabular cups do not benefit from the superior bearing surface and stability which is provided by the known larger type of prosthetic ball head and socket arrangements described above.

### SUMMARY OF THE INVENTION

It is therefore an object of the present invention to provide a method to successfully attach a large-sized ceramic acetabular cup to the acetabulum for use with a large-sized ceramic femoral ball head.

Therefore, according to the present invention, an acetabular cup for use in a total hip prosthesis employing a natural sized femoral ball head comprises an inner bearing surface portion constructed from a ceramics material, an outer bone-interface layer which may be porous, and an outer coating of a bone growth stimulant substance applied to the outer bone-interface layer.

According to the second aspect of the present invention, a total hip prosthesis employing a natural sized femoral ball head comprises an acetabular component comprising an inner bearing surface portion constructed from a ceramics material, an outer bone-interface layer, and an outer coating of the bone growth stimulant substance applied to the outer bone-interface layer.

In the preferred construction the inner bearing surface portion is constructed from alumina, or zirconia toughened alumina (ZTA). The outer bone-interface layer can be made from any suitable material, for example a metal or a porous ceramics material. Titanium can be used because there is a large amount of data available on its bone-interfacing performance. If desired a porous alumina can be used because it allows for bone-growth into the cavities in the material, resulting in a firm sphere-wide attachment. The bone growth stimulant can be any suitable compound such as bone morphogenic proteins. In a preferred embodiment, a hydroxyapatite coating is used. This material is known to be a successful bone-growth stimulant. Its presence on the outer bone-interface layer stimulates the bone to grow around or into the layer, holding it firmly in place.

The invention also includes a method of manufacturing an acetabular cup of the kind set forth above which includes the stages of: heating the bone interface layer; applying the bone interface layer to the inner bearing surface portion; and allowing the bone interface layer to shrink and form a secure fitting on the inner bearing surface portion. Thus, a titanium outer shell can be shrunk fit on to a ceramic acetabulum cup including the bearing surface.

Also included in the invention is a method of manufacturing a cup of the kind set forth above which includes the steps of: applying the alumina to the inner bearing surface portion by spraying; and allowing the alumina to set.

### BRIEF DESCRIPTION OF THE DRAWINGS

The present invention can be performed in various ways but two embodiments will now be described by way of example and with reference to the accompanying drawings in which:

FIG. 1 is a cross-sectional side view of an acetabular cup according to the present invention;

FIG. 2 is a cross-sectional side view of another acetabular cup according to the present invention; and

FIG. 3 is a cross-sectional side view of a total prosthetic hip joint implanted according to the present invention.

### DETAILED DESCRIPTION

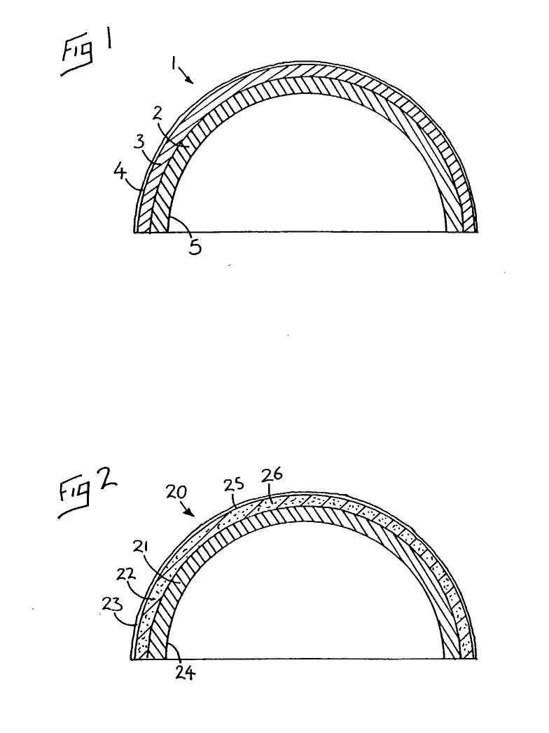

In FIG. 1 an acetabular cup according to the present invention 1 comprises an inner bearing surface portion 2, an outer bone-interface layer 3 and a hydroxyapatite coating 4. The inner bearing surface portion 2 has a bearing surface 5, and is constructed from a ceramics materials, for example dense ZTA. The outer bone-interface layer 3 is a titanium alloy support shell, for example made from TA6V, which has been attached to the inner ceramic bearing surface portion by shrinkage after heating during manufacture. (It can be attached in any other known way, for example brazing, gluing, taper lock or screw.) Thus a preferred method includes the steps of: heating the bone interface layer; applying the bone interface layer to the inner bearing surface portion; and allowing the bone interface layer to shrink and form a secure fitting on the inner bearing surface portion.

A compressive force can be applied between the interface layer and the inner bearing surface portion during shrinkage.

With this invention the surface finish of the bone interface layer can be structured, by machining or any other method, before the HA coating is applied.

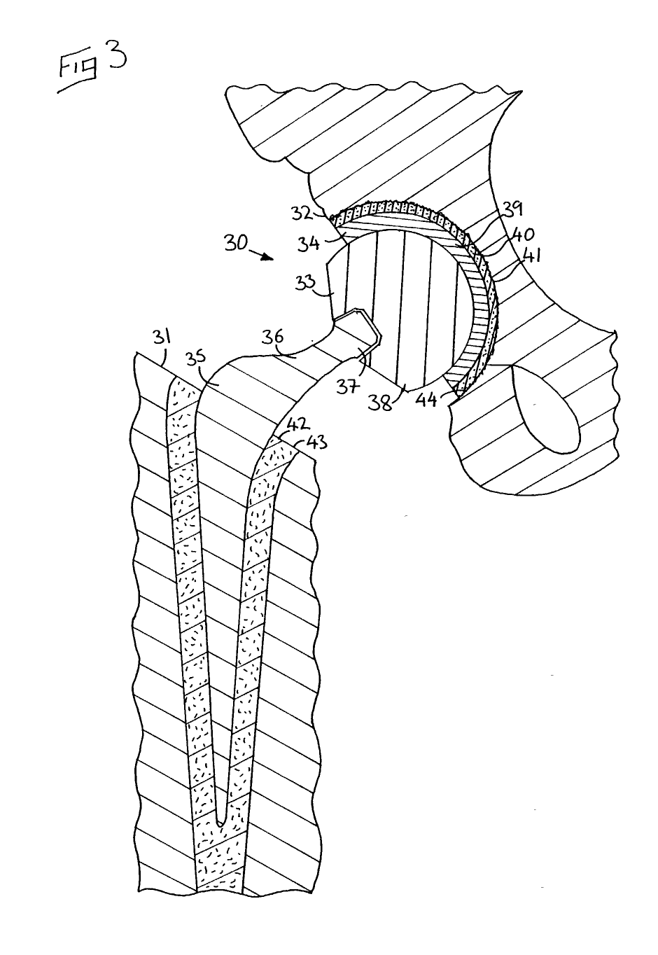

In FIG. 2, a second acetabular cup according to the present invention 20 comprises an inner bearing surface portion 21, an outer bone-interface layer 22 and a hydroxyapatite coating 23. The inner bearing surface portion has a bearing surface 24, and is constructed from a ceramics material. The outer bone-interface layer 22 is porous alumina, which has been sprayed on, for example by plasma spraying, or applied in any conventional way, during manufacture. The porous alumina has a rough surface 25 and cavities for bone growth in its body 26. In a preferred method the bone interface layer 22 is applied either by spraying or any other convenient method, onto a ceramics cup 21 before sintering. Then the cup and the porous layer are submitted to other finishing steps as any other massive ceramics product. Finally, a hydroxyapatite coating is sprayed onto the porous surface 23. Process for performing the coatings are well known in the art and are described in U.S. Pat. Nos. 4,746,532, 5,730,598 and 5,788,891, the teachings of which are incorporated herein by reference.

In both the acetabular cups in FIGS. 1 and 2, the preferred inner bearing surface portion can be between 3-5 millimeters thick, the preferred outer bone-interface layer can be between 0.1-2 millimeters thick and the preferred hydroxyapatite coating can be between 0.020 and 0.2 millimeters thick.

In FIG. 3, a total replacement hip joint according to the present invention 30 has been implanted into the femur 31 and the acetabulum 32. The hip joint 30 comprises a femoral stem component 33 and an acetabular cup component 34. The femoral stem component 33 comprises a body 35, a neck 36, a spigot 37 and a large ball head 38 made from alumina and mounted on said spigot 37. The acetabular cup component 34 comprises an inner bearing surface portion 39 constructed from a ceramics material, an outer bone-interface layer 40 made from porous alumina, and a hydroxyapatite coating 41.

As shown in FIG. 3, the femoral stem component 33 has been successfully implanted into the medullary canal 42 with the use of cement 43. The acetabular cup 34 has been successfully implanted into the acetabulum 32. The exposed bony matter of the acetabulum 44 has interfaced with the outer bone-interface layer 40 thanks to the hydroxyapatite bone growth stimulant 41. With this arrangement the multi-directional loading of the prosthetic hip joint will not result in prosthesis failure because the load will be carried across the sphere of the acetabular cup. Further, the ceramics on ceramics bearing provides smooth movement, and the large size of the joint provides stability during movement and increased security from dislocation.

In a third embodiment, the ceramic bearing portion is coated with sprayed alumina to form a porous surface while in the unfired (green) state. After spraying with alumina the composite structure is fired or sintered to form the ceramic implant. After firing, the composite structure may be coated with hydroxyapatite or other bone induction or conduction material.

Therefore, a large-sized ceramic acetabular cup is provided which can be successfully attached to the acetabulum for use with a large sized ceramic femoral head component.

Further, a total hip replacement joint is provided with the benefits of a natural-sized ceramics on ceramics bearing surface.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. An acetabular cup for use in a total hip prosthesis employing an anatomically sized femoral ball head comprising:
an inner bearing surface portion constructed from a ceramics material;
an outer bone-interface layer; and
an outer coating of a bone growth stimulant substance applied to the outer bone-interface layer.

2. The acetabular cup as claimed in claim 1 wherein the outer bone-interface layer is constructed from titanium, or a titanium alloy.

3. The acetabular cup as claimed in claim 2 wherein the titanium or titanium alloy has been attached to the inner bearing surface portion by shrinkage after heating during manufacture.

4. The acetabular cup as claimed in claim 1 wherein the outer bone-interface layer is constructed from porous alumina, provided with a rough outer surface and cavities for bone growth.

5. The acetabular cup as claimed in claim 4 wherein the porous alumina has been sprayed onto the inner bearing surface portion.

6. The acetabular cup as claimed claim 1 wherein the inner bearing surface portion is constructed from alumina.

7. The acetabular cup as claimed in claim 1 wherein the inner bearing surface portion is constructed from zirconia toughened alumina.

8. The acetabular cup as claimed in claim 1 wherein the bone growth stimulant substance is a hydroxyapatite coating.

9. The acetabular cup as claimed in claim 1 wherein the inner bearing surface portion is between 3 and 5 millimeters thick.

10. The acetabular cup as claimed in claim 1 wherein the outer bone interface layer is between 0.1 and 2 millimeters thick.

11. The acetabular cup as claimed in claim 1 wherein the hydroxyapatite coating is between 0.020 and 0.2 millimeters thick.

12. A method of manufacturing an acetabular cup for use in a total hip prosthesis employing an anatomically sized femoral ball head which comprises an inner bearing surface portion constructed from a ceramics material, an outer bone-interface layer constructed from titanium or a titanium alloy, and an outer coating of a bone growth stimulant substance applied to the outer bone-interface layer comprising:
heating the bone interface layer;
applying the bone interface layer to the inner bearing surface portion; and
allowing the bone interface layer to shrink and form a secure fitting on the inner bearing surface portion.

13. The method of manufacturing an acetabular cup for use in a total hip prosthesis employing an anatomically sized femoral ball head as claimed in claim 12 which includes applying a compressive force between the interface layer and the inner bearing surface portion during shrinkage.

14. A method of manufacturing an acetabular cup for use in a total hip prosthesis employing an anatomically sized femoral ball head which comprises an inner bearing surface portion constructed from a ceramics material, an outer bone-interface layer constructed from porous alumina, and an outer coating of a bone growth stimulant substance applied to the outer bone-interface layer comprising:
applying the alumina to the inner bearing surface portion by spraying; and
firing the inner bearing coated with alumina.

15. A method of manufacturing an acetabular cup for use in a total hip prosthesis employing an anatomically sized femoral ball head which comprises a ceramic inner bearing surface portion constructed from an unfired ceramics material, an outer bone-interface layer constructed from porous alumina, and an outer coating of a bone growth stimulant substance applied to the outer bone-interface layer, comprising:
applying the alumina to the unfired ceramic inner bearing portion by spraying; and
firing the inner bearing coated with alumina.

16. The method of maintaining an acetabular cup as set forth in claim 15, further comprising applying the outer bone-interface layer to the fired cup.

17. The method as set forth in claim 16 wherein the outer bone-interface layer is hydroxyapatite.
